(19) United States
(12) Patent Application Publication
(10) Pub. No.: US 2024/0295556 A1
(43) Pub. Date: Sep. 5, 2024

(54) METHOD FOR SIMULTANEOUSLY DIAGNOSING ERWINIA CAROTOVORA AND PHYTOPHTHORA INFESTANS IN SOIL BY USING SEMI-QUANTITATIVE LATERAL FLOW IMMUNODIAGNOSTIC TECHNIQUE

(71) Applicants: VETALL LABORATORIES, Gimpo-si (KR); ABC CIRCLE CO., LTD., Chungcheongbuk-do (KR)

(72) Inventors: Jihyun KIM, Gimpo-si (KR); Miyoung YANG, Incheon (KR); Jeongmi KIM, Goyang-si (KR); Inseo PARK, Hwaseong-si (KR); Jaejun LEE, Osan-si (KR)

(21) Appl. No.: 18/270,659

(22) PCT Filed: Jan. 3, 2022
(86) PCT No.: PCT/KR2022/000001

§ 371 Date: Jun. 30, 2023

(30) Foreign Application Priority Data
Jan. 4, 2021 (KR) 10-2021-0000507

(51) Int. Cl. G01N 33/569; G01N 33/543

(52) U.S. Cl. CPC G01N 33/56961; G01N 33/54388; G01N 33/56911; G01N 2333/195; G01N 2333/37; G01N 2469/10

(57) ABSTRACT

The present invention provides a simultaneous diagnosis method and a simultaneous diagnosis kit for Erwinia carotovora and Phytophthora infestans in the soil using semi-quantitative lateral flow immunoassay, for early identification and prevention of causative organisms that may cause a plant soft rot disease and a plant blight disease in the soil around a plant growth environment using semi-quantitative lateral flow immunoassay.

### TECHNICAL FIELD

The present invention relates to a simultaneous diagnosis method and a simultaneous diagnosis kit for Erwinia carotovora and Phytophthora infestans in the soil using semi-quantitative lateral flow immunoassay. More specifically, the present invention relates a method for simultaneously diagnosing pathogens present in the soil in advance before the pathogens infect plants and a simultaneous diagnosis kit capable of measuring semi-quantitatively the density of the pathogens by including rat monoclonal antibodies that specifically bind to Erwinia carotovora (Erwinia) causing a bacterial soft rot disease and Phytophthora infestans (Phytophthora) causing a fungal blight disease in plants, which are difficult to be accurately diagnosed due to the redundancy of onset times and the similarity of onset symptoms.

### BACKGROUND ART

Erwinia carotovora (Erwinia), which causes a bacterial plant soft rot disease, and Phytophthora infestans (Phytophthora), which causes a fungal plant blight disease are known to be lethal plant pathogens and are present in the soil before infecting plants.

The two types of pathogens present in the soil infect plants through a plant soil surface, and are one of the most damaging diseases in most farmhouse horticultural crops, and since the time and symptoms occurring in plants are similar, clear prescriptions based on accurate diagnosis have not practically been made in the farmhouse. However, the farmhouse depends on its own experience to determine a disease, but due to this inaccurate diagnosis, multiple types of bacterial disease and fungal disease agents are mixed and sprayed, and sprayed at high concentrations above a standard amount, which causes environmental pollution as well as the increased management cost in the farmhouse. In addition, the two types of pathogens have a fast disease progression rate, so that the transition to other plants proceeds very rapidly, and as a result, if the treatment period is missed before the plant is infected or at the early stage of infection even if it is infected, the pathogens quickly reach the developmental stage, and thus it is difficult to prevent disease even with chemical pesticides, causing a decrease in yield of crops, and also directly causing serious damage to the farmhouse by dying crops.

Therefore, methods for effectively controlling the two types of pathogens have been studied. For example, in Korean Patent Publication No. 10-2018-0117322 (Patent Document 1), as the title of a novel Serratia sp. IM2 strain, and a composition for plant disease control comprising the same, there is disclosed “a composition for controlling one or more plant diseases selected from the group consisting of bacterial spot disease and soft rot disease comprising a Serratia sp. IM2 strain (Accession No. KACC 92171P) or its culture medium as an active ingredient”.

In Korea Patent Publication No. 2002-0050672 (Patent Document 2), as the title of a new Streptomyces sp. AG-P (KCTC 8965P), and an agent for controlling plant diseases using the same, and there is disclosed an agent for controlling plant diseases exhibiting “an antibacterial activity against Phytophthora infestans and an inhibitory effect of a bacterial culture medium on the mycelial growth of Pythium sp. which is Rhizoctonia in vitro according to a dilution ratio of Streptomyces sp. AG-P (KCTC 8965P) strain culture medium”.

However, most of the prior arts including the inventions of the patent documents relate to technologies for treating diseases that have already occurred by killing or inhibiting the causative organisms causing plant soft rot disease and blight disease. There are almost no proposals for methods and tools for diagnosing the infection and severity of the causative organisms causing these specific bacterial soft rot disease and fungal blight disease before plants are infected or even at the early stage of infection even if it is infected. However, in Korean Patent Publication No. 1999-0079605 (Patent Document 3), as the title of a primer for PCR diagnosis of vegetable soft rot disease bacteria and there is disclosed “preparation of a primer for rapid and accurate diagnosis of soft rot disease pathogens by a PCR process by analyzing a novel gene fragment pECC2F (plasmid Erwinia carotovora subsp. carotovora 2F) present specifically in Erwinia carotovora subsp. carotovora, which is a bacterium causing a soft rot disease in vegetable crops to determine a nucleotide sequence of the gene, and using the nucleotide sequence of pECC2F”. In Korean Patent Publication No. 10-0736797 (Patent Document 4), as the title of a SCAR primer set capable of discriminating a mating type of Phytophthora infestans, there is disclosed “a sequence characterized amplified region (SCAR) primer set capable of amplifying a specific DNA fragment only in an A1 mating type of Phytophthora infestans using a polymerase chain reaction (PCR) technique”

However, the methods shown in Patent Documents 3 and 4 above detect and identify pathogenic and non-pathogenic organisms by complex steps of a gene amplification method, and it is practically meaningless to diagnose the soft rot disease and the blight disease described above, which frequently occur in the farmhouse, using the methods. Accordingly, the two types of diseases may be diagnosed more simply and quickly and preferably simultaneously before the plants are infected or even at an early stage of the infection even if it is infected, and furthermore, there has been a continuous demand for a practical method capable of quantitative diagnosis and a simultaneous diagnosis kit capable of realizing this method.

Meanwhile, in Korean Patent Publication No. 10-2154628 (Patent Document 5), as the title of a simultaneous diagnosis method of Ralstonia solanacearum and Fusarium oxysporum using semi-quantitative lateral flow immunoassay, there is disclosed a “semi-quantitative and simultaneous disease diagnosis method and mouse monoclonal antibodies”.

However, the method shown in Patent Document 5 above can quantitatively diagnose the determination and extent of pathogens by using the soil surface of a diseased plant as a sample, but there is no great effect or difficulty in preventing or monitoring pathogens before infecting the plants.

Because, first, in the case of soil diseases such as Erwinia carotovora and Phytophthora, symptoms similar to other types of bacteria that occur in the soil, such as yellowing, browning, and tissue necrosis are shown, thereby making it difficult to be diagnosed accurately, and cases of symptoms vary depending on a host crop and a season, thereby making it difficult to actually make an accurate diagnosis. In addition, infectious pathogenic microorganisms spread widely in a wide range within 1 week under optimal conditions, and thus, in the case of existing methods of mechanical diagnosis or inspection of the soil surface samples, it can be said that it is very unrealistic because it takes a long diagnosis time of 2 weeks or more and high cost due to various pretreatment processes, and culture and identification processes. In addition, due to the need for professional manpower and expensive equipment, it is difficult to apply in a wide range unlike developed patent products, and thus its effectiveness is very low.

In addition, in the case of an empirical diagnosis by symptomatic presentation by experts in a target soil disease, it is difficult to control the disease as a pesticide having most of the preventive effects because it is determined by symptoms after crop infection rather than a preventive method, and the effect thereof is not significant.

In addition, in the production of mouse monoclonal antibodies in Patent Document 5, mice are generally used to prepare hybridoma cells that produce monoclonal antibodies against antigens of various species. However, when studies on specific nucleotide sequences or antigenic sites have not been performed, such as the bacteria and fungi, a mouse monoclonal antibody prepared using an inactivated pathogen itself as an immunogen may exhibit high specificity, but it is not suitable for diagnosing pathogens in the early stage of infection because it is difficult to prepare antibodies that show high sensitivity that recognizes specific epitopes present in trace amounts of pathogens.

In addition, in Patent Document 5, a mouse ascites production method is used in the mass production method of antibodies, and this mouse ascites production method showed high production yield and convenience, but there are a possibility of non-specific binding by a mouse-derived endogenous antibody and a possibility that a difference in performance between antibodies may occur depending on the number of repeated production.

### PRIOR ARTS

### Patent Documents

(Patent Document 1) Korean Patent Publication No. 10-2018-0117322

(Patent Document 2) Korean Patent Publication No. 2002-0050672

(Patent Document 3) Korean Patent Publication No. 1999-0079605

(Patent Document 4) Korean Patent Publication No. 10-0736797

(Patent Document 5) Korean Patent Publication No. 10-2154628

### DISCLOSURE

### Technical Problem

The present invention has been invented considering the technical problems in the prior art, and a main object of the present invention is to provide rat monoclonal antibodies that specifically bind to Erwinia carotovora and Phytophthora infestans for first detection in the soil before plants are infected with bacterial or fungal causative organisms that cause a bacterial soft rot disease and a fungal blight disease in plants with high specificity and sensitivity. Therefore, an object of the present invention is to provide a simultaneous diagnosis method for Erwinia carotovora and Phytophthora infestans in the soil using semi-quantitative lateral flow immunoassay, capable of simultaneously measuring the potencies of these pathogens present in the soil in a semi-quantitative manner using the rat monoclonal antibodies that specifically bind to Erwinia carotovora and Phytophthora infestans.

Another object of the present invention is to provide a simultaneous diagnosis kit capable of simultaneously measuring the infection degree of pathogens semi-quantitatively using antibodies that specifically bind to Erwinia carotovora and Phytophthora infestans.

### Technical Solution

The present applicants recognized the problems in the art as described above, particularly provided a simultaneous diagnosis method using semi-quantitative lateral flow immunoassay so as to solve the problems of conventional inaccurate diagnosis and difficulty of rapid analysis for the two types of bacterial soft rot disease and fungal blight disease, and thus, prepared a standard for quantitative measurement by analyzing the potency of pathogens causing the diseases in plants by proliferating the pathogens present in the soil.

In addition, in the development of antibodies, since rats may provide a greater number of spleen B cells than mice to exhibit a better ability to recognize epitope regions for small or low immunogenic antigens, such rat monoclonal antibodies may allow higher sensitivity to a target than mouse monoclonal antibodies.

In addition, in a mass production method of antibodies, when a serum-free culture method was used, the reproducibility of production may be enhanced as compared to a mouse ascites production method, and an endogenous antibody capable of exhibiting non-specific binding was not included to secure an antibody with higher specificity.

Accordingly, research was conducted on semi-quantitative lateral flow immunoassay for a soft rot disease and a blight disease of enabling scientific and accurate diagnosis in the above manner and methods for development and mass production of antibodies that can be used therefor, and prevention and monitoring of diseases, and then the present invention was completed.

To achieve the object, one aspect of the present invention provides a rat monoclonal antibody exhibiting a specific immune response to Erwinia carotovora (Erwinia) causing a bacterial soft rot disease and Phytophthora infestans (Phytophthora) causing a fungal blight disease in plants.

The present invention may provide a simultaneous diagnosis method of Erwinia and Phytophthora using semi-quantitative lateral flow immunoassay, configured by steps of providing a primary antibody for capturing each antigen, a detection label, and a secondary antibody bound to the detection label, and binding Erwinia and Phytophthora samples to the respective antibodies, and quantifying the amount of each of the bound pathogens, and provide two types of diagnostic strips capable of detecting Erwinia and Phytophthora in one test kit.

In addition, according to another configuration of the present invention, the simultaneous diagnosis kit may diagnose pathogens semi-quantitatively by binding antibodies specific to each pathogen and comparing colors of the reaction with a control line to determine the amounts of pathogens in the sample as high potency, medium potency, low potency, and negative. At this time, the semi-quantitative simultaneous diagnosis kit determines low potency: 10⁴ cfu/g, medium potency: 10⁵ cfu/g, and high potency: 10⁶ cfu/g in the case of Erwinia, and low potency: 10³ cfu/g, medium potency: 10⁴ cfu/g, and high potency: 10⁵ cfu/g in the case of Phytophthora, as the step of quantifying the amounts of Erwinia and Phytophthora present in the sample, respectively.

In addition, the simultaneous diagnosis kit includes a primary antibody for capturing bacterial Erwinia carotovora and fungal Phytophthora infestans antigens, a detection label, a secondary antibody bound with the detection label, and a reagent for measuring the activity of the detection label, and the primary antibody and the secondary antibody for capturing the antigens may be used with a rat anti-Erwinia 5E10 antibody gold conjugate and a rat anti-Phytophthora 1C6 antibody gold conjugate obtained in the step of providing the rat monoclonal antibodies to Erwinia and Phytophthora of the simultaneous diagnosis method of the present invention.

According to yet another configuration of the present invention, the sample used in the diagnosis kit may be a soil diluent.

### Advantageous Effects

According to the present invention, it is possible to prevent infection of farm-grown plants in advance or minimize the damage by using a semi-quantitative lateral flow immunoassay kit containing antibodies showing high sensitivity and specificity to Erwinia and Phytophthora to determine pathogens before the causative organisms that cause a bacterial soft rot disease and a fungal blight disease of plants proliferate in the soil and infect plants.

Further, since the simultaneous diagnosis kit and the simultaneous diagnosis method for the soft rot disease and the blight disease in the plants may semi-quantitatively analyze the infection source within 10 minutes, it is possible not only to diagnose and determine the infection and type of disease-causing pathogens in plants at an early stage, easily and at a low cost, as compared to conventional uncertain visual diagnosis, microbial isolation and identification, and PCR analysis, but also to select the exact type and amount of therapeutic agent at the farmhouse by enabling semi-quantitative analysis and easily measuring and determining the degree of pathogens.

### DESCRIPTION OF DRAWINGS

FIG. 1 is a graph as an ELISA result showing the sensitivity and cross-reactivity with similar pathogens of a rat anti-Erwinia monoclonal antibody and a rat anti-Phytophthora monoclonal antibody finally prepared according to the present invention.

FIG. 2 is a representative diagram of a kit for detecting Erwinia and Phytophthora according to the present invention.

FIGS. 3A and 3B are schematic diagrams schematically illustrating a structure of a strip constituting a kit for detecting Erwinia and Phytophthora according to the present invention, respectively.

FIG. 4 is a photograph showing a state before using the kit for detecting Erwinia and Phytophthora according to the present invention.

FIG. 5 is a photograph showing results of testing exemplary samples for each potency using the kit in the kit for detecting Erwinia and Phytophthora according to the present invention.

FIGS. 6A and 6B are tables showing the interpretation according to the results of the kit for detecting Erwinia and Phytophthora according to the present invention, respectively.

### MODES OF THE INVENTION

According to the present invention, there is provided a diagnosis kit capable of quickly and easily identifying and determining Erwinia carotovora and Phytophthora infestans pathogens simultaneously within 10 minutes of testing time by preparing rat monoclonal antibodies that specifically bind to Erwinia carotovora and Phytophthora infestans and using semi-quantitative lateral flow immunoassay-based technology using these antibodies. Accordingly, it is possible to simultaneously measure the potencies of these pathogens present in the soil in a semi-quantitative manner by using the rat monoclonal antibodies that specifically bind to Erwinia carotovora and Phytophthora infestans.

More specifically, it is possible to prevent diseases in advance by determining the proliferation of pathogens capable of causing a bacterial plant soft rot disease and a fungal plant blight disease in the soil environment around plants using semi-quantitative lateral flow immunoassay and measuring the density of the pathogens in the soil in the semi-quantitative manner.

Hereinafter, the present invention will be described in more detail by preferred embodiments with reference to the accompanying drawings. However, it goes without saying that the scope of the present invention is not limited thereto.

That is, the embodiments are provided to make the disclosure of the present invention complete and completely announce the scope of the present invention to those skilled in the art to which the present invention belongs and the present invention is just defined by the scope of the claims. Thus, in some embodiments, well-known components, well-known operations and well-known techniques have not been described in detail in order to avoid obscuring the interpretation of the present invention.

It is also to be understood that the terminology used herein is for the purpose of describing embodiments only and is not intended to limit the present invention. In this specification, singular expressions used herein include plurals expressions unless otherwise particularly mentioned in the context. Also, components and operations referred to as ‘comprising (or including)’ do not exclude the presence or addition of one or more other components and operations.

As used in the present invention, the term “Erwinia carotovora (Erwinia)” pathogen is a gram-negative rod-shaped anaerobic bacterium, and proliferates well at an optimum temperature for growth of 27 to 30° C. and proliferates well when the soil is neutral or weakly alkaline and humid. Erwinia carotovora was distributed within 15 cm from the topsoil, but may survive at depths of 25 cm or higher, invaded various vegetables and plants, including ornamental plants such as carrots, potatoes, cucumbers, onions, tomatoes, lettuce and red vegetables, through wounds caused by soil pests or nematodes to cause diseases, and excessive use of nitrogen fertilizers may promote disease development. When Erwinia infects the plants, Erwinia produces a pectin enzyme and a plant cell wall lytic enzyme such as cellulose and invades the plants, and then the plant tissue becomes brittle and rots, causing a bad smell.

As used herein, the term “Phytophthora infestanse (Phytophthora)” pathogen is a type of oomycete that causes a serious disease called blight disease on potatoes and tomatoes. Spores of Phytophthora proliferate on leaves in the case of potato, and rapidly proliferate in a humid state of 12 to 18° C. and spread throughout the crops. Since the spores are vulnerable to high or low temperatures, hyphae and asexual sporangia may survive in plants or soil only for a short period of time, but may infect tubers and get through winter.

According to a preferred embodiment of the present invention, a simultaneous diagnosis method for Erwinia carotovora and Phytophthora infestans using semi-quantitative lateral flow immunoassay of the present invention may be configured by providing a rat monoclonal antibody of each of Erwinia carotovora (Erwinia) causing a bacterial soft rot disease and Phytophthora infestans (Phytophthora) causing a fungal blight disease; binding Erwinia and Phytophthora to the respective antibodies; and quantifying the bound pathogens.

According to another preferred embodiment of the present invention, in the simultaneous diagnosis method for Erwinia carotovora and Phytophthora infestans using semi-quantitative lateral flow immunoassay of the present invention, in the quantifying of the amounts of Erwinia and Phytophthora, in the case of Erwinia, it may be determined as low potency: 10⁴ cfu/g, medium potency: 10⁵ cfu/g, and high potency: 10⁶ cfu/g, and in the case of Phytophthora, it may be determined as low potency: 10³ cfu/g, medium potency: 10⁴ cfu/g, and high potency: 10⁵ cfu/g, in the case of Erwinia, 10³ cfu/g or lower may be tested negative, and in the case of Phytophthora, 10² cfu/g or lower may be tested negative.

According to yet another preferred embodiment of the present invention, the antibodies specific to Erwinia or Phytophthora are not particularly limited and may be selected as long as the antibodies are antibodies that specifically recognize each pathogen, but it is preferable to select a monoclonal antibody that is not cross-reactive with bacteria or fungi that may infect plants.

Further, the present invention provides a semi-quantitative test kit for determining causative organisms and analyzing the degree of infection by detecting pathogens present in the soil that may cause a soft rot disease or blight disease, using rat monoclonal antibodies that specifically react to Erwinia and Phytophthora, respectively.

In order to achieve the object of the present invention, the present invention uses each of a pathogenic bacterium Erwinia and a pathogenic fungus Phytophthora as immunogens for producing antibodies. The bacteria and fungi used in the present invention were introduced and cultured from the Korean Agricultural Culture Collection (KACC) through ABC Circle Co., Ltd., and the cultured bacteria and fungi were inactivated at a high temperature and used as immunogens for producing antibodies.

In order to achieve the object, in the present invention, rats (SD-Rat; Sprague Dawley Rat, 4 Weeks, Female) were immunized using inactivated bacteria and inactivated fungi as immunogens, and rat splenocytes and myeloma cells (Sp2/0 Ag-18) were cell-fused to prepare hybridomas. Then, monoclonal antibodies specific for Erwinia and Phytophthora were obtained from the prepared hybridomas, respectively. The sensitivity for the immunogens of the two rat anti-Erwinia monoclonal antibodies and the two rat anti-Phytophthoramonoclonal antibodies finally selected from the obtained antibodies is provided as the ELISA result, and the reactivity with 10 types of bacteria and fungi that may be present in infected plants targeted by the diagnosis kit or particularly in the soil and may cause cross-reactivity is provided as the ELISA result.

At this time, in the fungal species, in the case of the blight disease, the main crops are pepper, tomato, etc., and the host crops are similar to Fusarium, Ralstonia diagnosis kits, but in the case of the soft rot disease, there are cases of cultivation in open soil, such as Chinese cabbage and lettuce, and then tests are conducted by adding fungal species (Mycosphaerella nawae, etc.) that may be found in open soil.

In the present invention, rats are used in the development of antibodies. Since the rats may provide a greater number of spleen B cells than mice to exhibit a better ability to recognize epitope regions for small or low immunogenic antigens, such rat monoclonal antibodies may enable higher sensitivity to a target than mouse monoclonal antibodies.

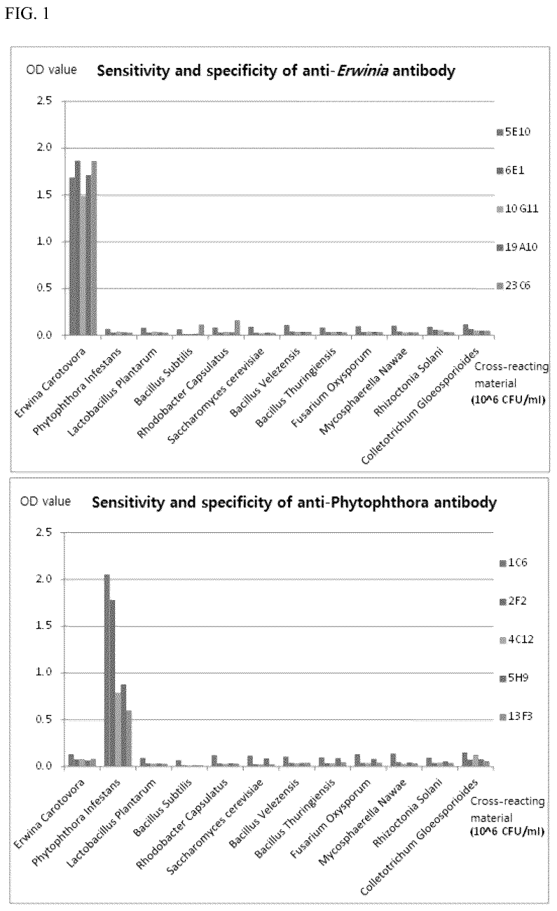

In order to achieve the object, the finally selected two rat anti-Erwinia antibodies and two anti-Phytophthoraantibodies showed high sensitivity to Erwinia carotovora and Phytophthora infestans and did not show cross-reactivity with each other. In addition, referring to FIG. 1, it was confirmed that all the two antibodies did not exhibit cross-reactivity to 6 types of bacteria (Lactobacillus plantarum, Bacillus subtillis, Rhodobacter capsulatus, Saccharomyces cerevisiae, Bacillus velezensis, Bacillus thuringiensis) and 4 types of fungi (Fusarium oxysporum, Mycosphaerella nawae, Rhizoctonia solani, Colletotrichum gloeosporioides). It was finally confirmed that 4 types of antibodies prepared according to the present invention specifically recognize each of Erwinia and Phytophthora, and accordingly, in the present invention, the antibodies are used for preparing the simultaneous diagnosis kit according to the present invention.

In order to achieve the object, the present invention provides assay strips capable of capturing pathogens from soil samples using rat monoclonal antibodies that specifically react to each of Erwinia and Phytophthora and specifically detecting each of Erwinia and Phytophthora in samples by rapid immunochromatography.

In order to achieve the object, the present invention provides a diagnosis method including injecting a predetermined amount of a sample into a contact area of the assay strip, forming a complex by combining a detection reagent (gold conjugate) provided with a predetermined label with an analyte in the sample, deploying the complex on a membrane, and detecting the pathogen by observing an appearance change of a reaction part having a stationary phase for a pathogen in a predetermined area on the membrane.

The diagnosis method of the present invention includes a sandwich assay or a competition assay.

In addition, the present invention relates to a simultaneous diagnosis kit for implementing the diagnosis method, including a primary antibody for capturing Erwinia carotovora bacterial and Phytophthora infestans fungal antigens, a detection label, a secondary antibody bound with the detection label, and a reagent for measuring the activity of the detection label.

In addition, the primary antibody and the secondary antibody for capturing the antigens may be used with a rat anti-Erwinia 5E10 antibody gold conjugate and a rat anti-Phytophthora 1C6 antibody gold conjugate obtained in the step of providing the rat monoclonal antibody to each of Erwinia and Phytophthora described in the simultaneous diagnosis method of the present invention.

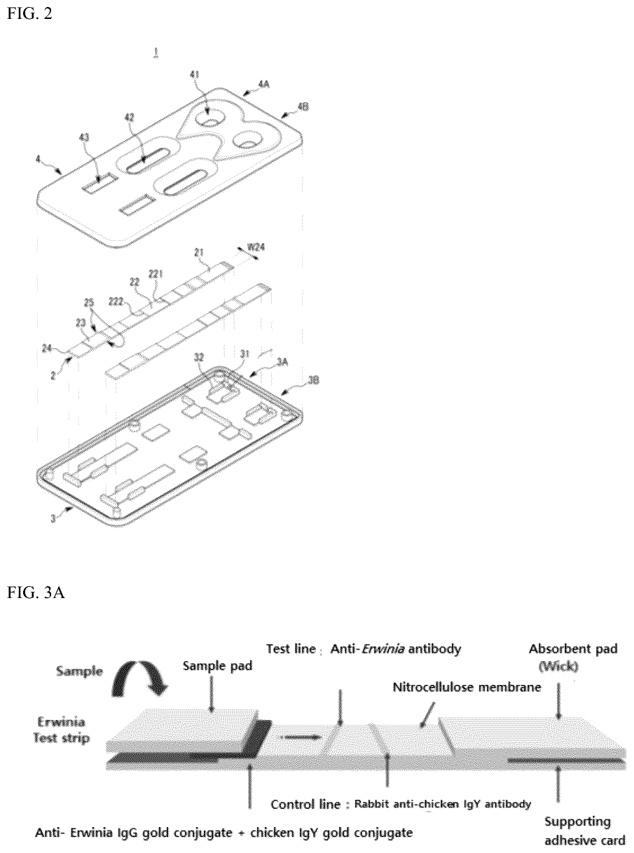

Referring to FIG. 2, more specifically, there is provided a diagnosis kit for detecting Erwinia and Phytophthora including an assay strip 2 in which a test line 221 having a stationary phase for an IgG antibody that specifically reacts to each of Erwinia and Phytophthora and a control line 222 for determining a normal operation are provided in a predetermined area on the membrane 22, and an immunoassay device provided with a sample inlet 41 for protecting the assay strip from various contaminants at all times and injecting at least a sample and a result display window 42 for observing reaction results in the test line 221 and the control line 222 on the assay strip.

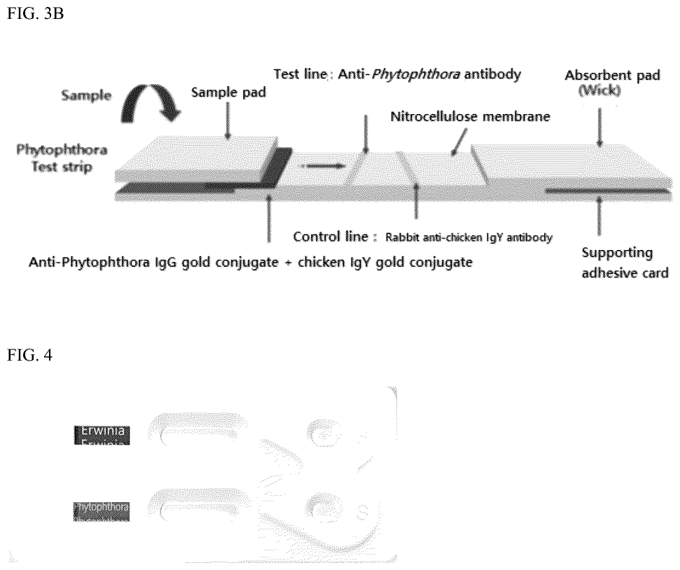

In order to detect Erwinia, a causative bacterium of the soft rot disease, and Phytophthora, a causative bacterium of the blight disease, from a soil sample by the immunochromatography, a specific antibody capable of detecting the pathogen is adsorbed on a nitrocellulose membrane at a predetermined location, and an antibody capable of selectively binding to the pathogen is conjugated to gold particles and dried on a pad. The dried gold conjugate pad and the pad applied with the sample are overlapped and covered on the nitrocellulose membrane, and an absorbent pad is included on an opposite side (see FIGS. 3A and 3B).

The membrane that may be used for preparing the assay strip according to the present invention may be used with materials for conventional diagnostic strips, and for example, one selected from various synthetic polymers such as nitrocellulose, cellulose, cellulose acetate, and polyethylene.

A labeled reagent that may be included in a control reagent may be applied in the same manner as the detection reagent. Examples of an auxiliary specific binding member are not particularly limited, but may be selected from, for example, avidin, biotin, FITC, anti-FITC antibody, rat immunoglobulin, and anti-rat immunoglobulin antibody.

The detection reagent includes a labeled reagent that allows the presence of an analyte to be tested to be identified from the outside through the naked eye or other instruments, an auxiliary specific binding member, or a component of a signal generating system. The common knowledge of the labeled detection reagent is already well known in the art to which the present invention pertains. Examples of such a label include a catalyst, enzymes (phosphatases, peroxidases), enzyme substrates (nitroblue tetrazolium, 3,5′,5,5′-tetranitrobenzidine, 4-methoxy- 1-naphthol, 4-chloro-1-naphthol, 5-bromo-4-chloro-3-indolylphosphate), a chemiluminescent enzyme substrate (dioxetane), fluorescent compounds (fluorescein, phycobiliprotein, rhodamine), a chemiluminescent compound, metal sol, non-metal sol, carbon sol, dye sol, particulate latex, color indicator, color materials included in liposomes, and the like.

The assay strip according to the present invention is put and prepared into a plastic single device (hereinafter referred to as an immunoassay device) indicated with the sample inlet 41 and the result display window 42 (see FIG. 2).

The sample is preferably used with a diluted soil sample solution. The soil sample is added and suspended into the dilution solution, precipitate for 5 minutes or more, and three drops of a supernatant are dropped onto a sample drop site of the sample inlet 41 using a disposable pipette. Erwinia or Phytophthora present in the sample is deployed on the nitrocellulose membrane 22 by capillary action while reacting with a specific antibody attached to the gold particle. The specific antibody capable of detecting Erwinia or Phytophthora, respectively, is adsorbed at a predetermined position on the test line 221 of the membrane. The pathogens present in the sample bind to the antibodies conjugated to gold particles to form a complex, and the complex binds to an antibody specific to Erwinia or Phytophthora located on the test line while passing through the test line to form a purple (red) band by the gold particle color at the corresponding position. Rabbit anti-chicken IgY was adsorbed to the position of the control line 222 so that chicken IgY-bound gold particles always reacted regardless of the presence or absence of pathogens in the sample to exhibit a purple (red) band.

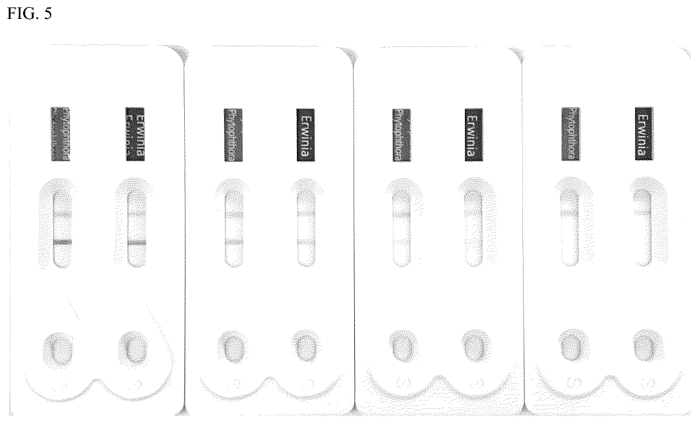

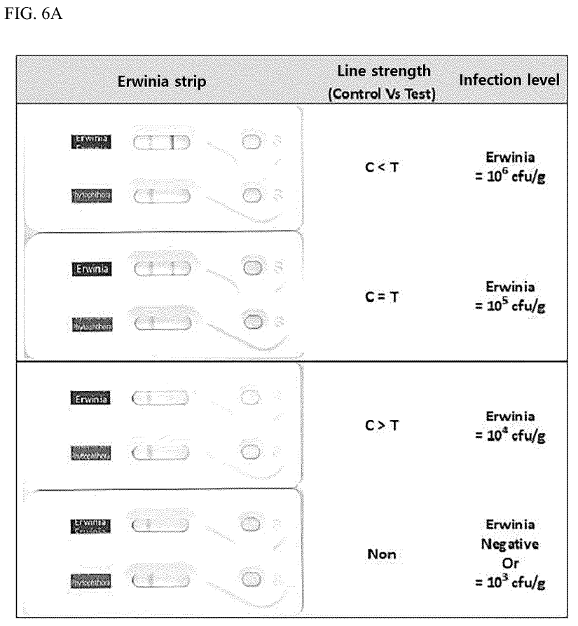

Non-reacting contents are permeated in the absorbent pad, and the membrane of the test window appears clean white to easily read the formed band. In addition, when Erwinia and Phytophthora do not exist in the sample, a purple or red band is formed only in the control line portion of the strip (see FIGS. 4 and 5).

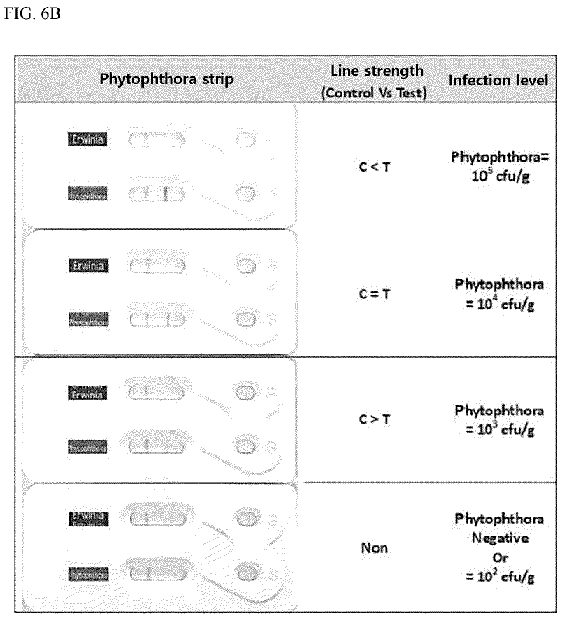

As a method of quantifying the presence and density of each of Erwinia and Phytophthora in the soil sample, the interpretation method for each result is described in detail in FIGS. 6A and 6B.

Hereinafter, raw materials, assay strips and a manufacturing method thereof according to the present invention will be described in more detail through the following Examples. However, these Examples are only presented to understand the contents of the present invention, and the scope of the present invention should not be construed as being limited to these Examples.

### Example 1

### Preparation of Rat Anti-Erwinia Antibody and Rat Anti-Phytophthora Antibody

### A. Preparation of Immunogenic Erwinia and Phytophthora Antigens

Bacteria and fungi used as rat immunogens were introduced and cultured from the Korean Agricultural Culture Collection (KACC) through ABC Circle Co., Ltd., and the cultured bacteria and fungi were inactivated at a high temperature and used as antigens for producing antibodies.

### B. Preparation of Hybridomas

Five hybridomas capable of producing antibodies that specifically reacted to causative organisms of each of a soft rot disease and a blight disease were prepared. Rats (SD-Rat; Sprague Dawley Rat, 4-week-old, female) were injected intraperitoneally with an emulsion in which inactivated Erwinia and Phytophthora were mixed with a complete adjuvant or incomplete adjuvant (Sigma), which is an immune enhancer, in a ratio of 1:1, respectively. Immunization was performed 6 times for 2 months, and rats with a high production rate of antibody in the blood were selected and the spleens were extracted from the selected rats. Among splenocytes obtained by crushing the spleens, only red blood cells were selectively removed using an RBC lysis buffer (Sigma). The splenocytes washed three times were mixed with myeloma cells (Sp2/0 Ag-18) at a ratio of 5:1, and mixed with 1 ml of PEG1500 (Polyethylene Glycol 1500, Sigma) to induce cell fusion. Hybridomas prepared by fusion of the two types of cells were suspended in a 96 well culture plate, and cultured for 1 week in a selective medium containing 10% fetal bovine serum (FBS, Hyclone) and an HAT media supplement (Sigma) in a DMEM (Hyclone) to remove unfused cells. The cells were cultured for 1 week in the culture medium containing 10% FBS and an HT media supplement (Sigma) in the DMEM and then the cell culture medium was collected and subjected to 1st screening such as ELISA, Western blot, isotyping, and the like to select clones producing antigen-specific antibodies. The selected clones were subjected to monoclonalization (isolation) and secondary screening, and then final monoclonal hybridomas were obtained.

C. Screening of Specific Antibodies

In the screening of a rat anti-Erwinia antibody and a rat anti-Phytophthora antibody, an antibody exhibiting high sensitivity to an antigen was selected, and an antibody without showing reactivity to a cross-reacting material was selected. The sensitivity and specificity of the antibody were confirmed through the ELISA method.

In the method for screening antibodies that meet these conditions, the reactivity to 10 types of bacteria (Lactobacillus plantarum, Bacillus subtillis, Rhodobacter capsulatus, Saccharomyces cerevisiae, Bacillus velezensis, and Bacillus thuringiensis) and 4 types of fungi (Fusarium oxysporum, Mycosphaerella nawae, Rhizoctonia solani, and Colletotrichum gloeosporioides) was confirmed by an ELISA method. The 6 types of bacteria and the 4 types of fungi were introduced, cultured and provided by ABC Circle Co., Ltd.

As a result of the test, the anti-Erwinia antibody had high sensitivity to Erwinia, and 5 types of antibodies without cross-reactivity to 10 types of bacteria and fungi were selected and obtained (see FIG. 1).

In addition, the anti-Phytophthora antibody had high sensitivity to Phytophthora, and 5 types of antibodies without cross-reactivity to 7 types of bacteria and fungi were selected and obtained (see FIG. 1).

### D. Mass Obtaining of Antibodies

In order to mass-produce antibodies from the obtained hybridomas, a serum-free culture medium production method was used. Serum free media without containing animal-derived serum were used for the serum-free culture medium in which the hybridomas were cultured, sodium hypoxanthine and thymidine, which may be used in a de novo biosynthesis pathway of nucleoside, as additives and penicillin-streptomycin as an antibiotic were used, and amino acids, vitamins, glucose, glutamine, trace elements, growth factors, insulin, transferrin, selenium, pyruvate, and the like were used as nutritional supplements. The hybridomas were suspension-cultured in a serum-free culture medium containing the additives at 37° C. in a 5% CO₂ environment, and the culture medium in which 70% or more of the cells were killed was recovered.

The antibodies were purified using a protein G resin (HiTrap Protein G HP column, GE). The purified monoclonal antibodies were obtained, and antibodies that specifically reacted with each of Erwinia and Phytophthora, and without exhibiting cross-reactivity to other bacteria and fungi were finally selected by conducting a sandwich pair test thereto. Finally, as shown in Table 1, a rat monoclonal antibody showing high sensitivity and specificity to Erwinia and a rat monoclonal antibody showing high sensitivity and specificity to Phytophthora were obtained, and used for the manufacture of a simultaneous diagnosis kit according to the present invention.

TABLE 1
Mass-production of rat anti-Erwinia antibody and rat anti-
Phytophthora antibody using serum-free culture method

| | |
|---|---|
| Mass-production of rat | Mass-production of rat |
| anti-Erwinia antibody | anti-Phytophthora antibody |

| | Antibody Yield/ | | Antibody Yield/ |
|---|---|---|---|
| Clone | Culture medium | Clone | Culture medium |
| 5E10 | 7.5 mg/1 L | 1C6 | 9.1 mg/1 L |
| 6E1 | 9.0 mg/1 L | 2F2 | 7.5 mg/1 L |
| 10G11 | 10.5 mg/1 L | 4C12 | 12 mg/1 L |
| 19A10 | 5.5 mg/1 L | 5H9 | 5.1 mg/1 L |
| 23C6 | 7 mg/1 L | 13F3 | 8 mg/1 L |

As shown in Table 1, when the serum-free culture method was used, the reproducibility of production may be enhanced as compared to a mouse ascites production method, and an endogenous antibody capable of exhibiting non-specific binding was not included to secure a pure antibody with higher specificity.

### Example 2

### Preparation of Detection Strips and Simultaneous Diagnosis Kit for Erwinia and Phytophthora

A. Preparation of Membrane Coated with Rat Anti-Erwinia Antibody and Rat Anti-Phytophthora Antibody

5 types of rat anti-Erwinia antibodies 5E10, 6E1, 10G11, 19A10, and 23C6 and 5 types of rat anti-Phytophthora antibodies 1C6, 2F2, 4C12, 5H9, and 13F3 which were specific for each pathogen were used as a test line at a final concentration of 1 mg/ml, and as a control line, rabbit anti-chicken IgY was used at a concentration of 1 mg/ml. The antibody of the test line and the solution of the control line were coated on a nitrocellulose membrane using a dispensing device (KINAMETICS, USA). The membrane was dried overnight in a low-humidity laboratory or fan-dried for at least 5 hours. A plate of the prepared membrane was stored in a sealed container with a desiccant or in a low-humidity laboratory.

### B. Preparation of Antibody-Gold Conjugates

An antibody-gold conjugate content test was performed by diluting monoclonal antibodies obtained to prepare antibody-gold conjugates of 5 types of rat anti-Erwinia antibodies 5E10, 6E1, 10G11, 19A10, and 23C6, and 5 types of rat anti-Phytophthora antibodies 1C6, 2F2, 4C12, 5H9, and 13F3, and optimal conjugation pH and antibody content conditions were selected. According to the concentrations of the antibodies selected above, the antibodies were added dropwise to a gold solution during stirring. After stirring each solution for 15 minutes, a 10% BSA solution was added to each gold particle suspension. After stirring for 15 minutes again, the bound gold solution was centrifuged, a supernatant was discarded to remove unbound antibodies, the bound gold solution (pellet) was added with 5 mM borate (sodium tetraborate, pH 7.2) with 1% BSA added three times the amount of the pellet, and then the pellet was resuspended. The suspension was centrifuged again, and the final pellet was prepared by adjusting the absorbance to 10±1 O.D. at 530 nm with a spectrophotometer in 5 mM borate (sodium tetraborate, pH 7.2) added with 1% BSA (see Table 2).

TABLE 2
Selection for gold conjugation conditions of
anti-Erwinia and anti-Phytophthora antibodies

| Anti-Erwinia antibody | | | Anti-Phytophthora antibody | | |
|---|---|---|---|---|---|
| gold conjugation | | | gold conjugation | | |
| | | Antibody | | | Antibody |
| Clone | pH | concentration | Clone | pH | concentration |
| 5E10 | pH 6.0 | 14 ug/ml | 1C6 | pH 7.0 | 20 ug/ml |
| 6E1 | pH 8.0 | 14 ug/ml | 2F2 | pH 6.0 | 18 ug/ml |
| 10G11 | pH 6.0 | 20 ug/ml | 4C12 | pH 8.0 | 12 ug/ml |
| 19A10 | pH 7.0 | 14 ug/ml | 5H9 | pH 6.0 | 16 ug/ml |
| 23C6 | pH 6.0 | 20 ug/ml | 13F3 | pH 6.0 | 16 ug/ml |

### C. Preparation of Chicken IgY Antibody Gold Conjugates

Chicken IgY (Fitzgerald, 70-B9093RA00-A0) was added dropwise to the gold solution while stirring to a final concentration of 20 ug/ml, and then the solution was stirred for 15 minutes again. Thereafter, a 10% BSA solution was added to the gold particle suspension. After stirring for 15 minutes again, the bound gold solution was centrifuged, a supernatant was discarded to remove unbound antibodies, the bound gold solution (pellet) was added with 5 mM borate (sodium tetraborate, pH 7.2) with 1% BSA added three times the amount of the pellet, and then the pellet was resuspended. The suspension was centrifuged again, and the final pellet was prepared by adjusting the absorbance to 10±1 O.D. at 530 nm with a spectrophotometer in 5 mM borate (sodium tetraborate, pH 7.2) added with 1% BSA.

D. Preparation of Gold Conjugate-Treated Pads

The gold conjugates prepared in B and C were prepared as follows by adding 5% trehalose. For Erwinia diagnostic strips, a rat anti-Erwinia antibody-gold conjugate at a final concentration of 3.0 O.D. (optical density) and a chicken IgY-gold conjugate at a final concentration of 0.5 O.D. were prepared in 0.5×20 cm glass fiber. For Phytophthora diagnostic strips, a rat anti-Phytophthora antibody-gold conjugate at a final concentration of 3.0 O.D. (optical density) and a chicken IgY-gold conjugate at a final concentration of 0.5 O.D. were prepared in 0.5×20 cm glass fiber.

### E. Preparation of Absorbent Pad and Sample Pad

An absorbent pad and a sample pad were dried and prepared so that the reaction solution was well absorbed.

### F. Device Assembly

From the right, in the membrane and the pads prepared above, a sample pad, a gold pad (gold conjugate-treated pad), a nitrocellulose membrane, and finally an absorbent pad were overlapped and bound with each other and cut in a strip size suitable for a size of an immunoassay device. The cut strips were finally put into the lower plate of the diagnostic immunoassay device, respectively, and covered with the upper plate to prepare a kit for simultaneous diagnosis.

### G. Antibody Pairing Test

In order to select an optimal sandwich pair for the selected antibodies, a pairing test functioning on the assembled rapid diagnosis kit was performed. Positive and negative reactions were confirmed for actual samples of 25 pairs of Erwinia and 25 pairs of Phytophthora, and one pair exhibiting high sensitivity and specificity by showing an optimal antigen-antibody reaction on a rapid diagnosis kit strip was finally selected, respectively. The selected Erwinia #10G11/#5E6-gold pair and Phytophthora #2F2/#1C6-gold pair exhibited the best sensitivity and specificity through optimization of strip conditions and a final product detectable by potency was produced.

### H. Configuration of Product

The immunoassay device described above, a sample diluent, a sample diluent bottle, and a dropper were configured as a final product.

### Example 3

### Selection of Cut-Off Levels of Pathogens According to the Present Invention

A. In the simultaneous diagnosis kit for Erwinia and Phytophthora prepared in Example 2, in order to select the cut-off levels of the pathogens, ABC Circle Co., Ltd. conducted a concentration investigation test in the soil of Erwinia causing a soft rot disease and Phytophthora causing a blight disease in plants. Seedlings of three host plants (tomato, cabbage, and potato) were planted and cultivated. Each of the cultured Erwinia and Phytophthora was inoculated with different pathogen densities in the soil where the plants were planted. Symptoms of soft rot disease and blight disease appearing on crops were confirmed under room temperature conditions, and the identification and densities of microorganisms in the surface soil around the plants were measured at 5-day intervals. As a result, when the initial symptoms of the soft rot disease were visually confirmed in plants cultivated in Erwinia challenge soil, the pathogen density was confirmed to be 1×10⁴ cfu/g or higher, and at 1×10⁵ cfu/g or higher, lesions of the soft rot disease were completely identified, and then the plants began to die. The density of Erwinia was 1×10³ cfu/g or lower in untreated and non-developed soils. In addition, when early symptoms of the blight disease were visually confirmed in plants cultivated in Phytophthora challenge soil, it was confirmed that the pathogen density was 1×10³ cfu/g or higher, and at 1×10⁴ cfu/g or higher, the blight disease progressed rapidly from 10 days after inoculation, and the plants began to die. At 1×10² cfu/g or lower of Phytophthora infestans, the onset phenotype was insignificant, making it impossible to determine the presence or absence of infection. The results of the pathogen inoculation and the concentration investigation test in the soil for the three host plants were shown in Tables 3 and 4.

TABLE 3
Density of Pathogenic Microorganisms in the soil on Erwinia Infection by Crop

| | At 5 day | | | | At 10 day | | | |
|---|---|---|---|---|---|---|---|---|
| | Density of pathogen (cfu/g) | | | | | | | |
| Crop | 1 × 10² | 1 × 10³ | 1 × 10⁴ | 1 × 10⁵ | 1 × 10³ | 1 × 10⁴ | 1 × 10⁵ | 1 × 10⁶ |
| Tomato | 0 | 0 | 1.3 | 2.3 | 0 | 1.3 | 3.5 | 3.7 |
| Chinese | 0 | 0 | 1.7 | 2.0 | 0.3 | 1.3 | 3.7 | 4.0 |
| cabbage | | | | | | | | |
| Potato | 0 | 0.3 | 1.7 | 2.3 | 0.3 | 1.7 | 3.7 | 4.0 |

* Disease ratings: 0 = No lesions, 1 = occasional lesions, 2 = lesions sparse but uniformly distributed, 3 = dense uniform distribution of lesion), 4 = complete blight

TABLE 4
Density of Pathogenic Microorganisms in the soil on Phytophthora Infection by Crop

| | At 5 day | | | | At 10 day | | | |
|---|---|---|---|---|---|---|---|---|
| | Density of pathogen (cfu/g) | | | | | | | |
| Crop | 1 × 10² | 1 × 10³ | 1 × 10⁴ | 1 × 10⁵ | 1 × 10³ | 1 × 10⁴ | 1 × 10⁵ | 1 × 10⁶ |
| Tomato | 0 | 1.7 | 3.7 | 3.7 | 1.5 | 3.5 | 4.0 | 4.0 |
| Chinese | 0 | 1.0 | 3.5 | 3.7 | 1.7 | 3.7 | 4.0 | 4.0 |
| cabbage | | | | | | | | |
| Potato | 0 | 1.5 | 4.0 | 4.0 | 1.7 | 3.7 | 4.0 | 4.0 |

*Disease ratings: 0 = No lesions, 1 = occasional lesions, 2 = lesions sparse but uniformly distributed, 3 = dense uniform distribution of lesion, 4 = complete blight

B. As described above, the cut-off levels of the pathogens in the Erwinia and Phytophthora diagnosis kits prepared according to the symptoms occurring in the soft rot disease and the blight disease and the density of the pathogen were selected, and in order to analyze the selected cut-off levels semi-quantitatively, the cut-off levels were determined as high, medium, and low potencies, which were shown in Table 5 below.

TABLE 5
Selection of cut-off levels for Erwinia
and Phytophthora diagnosis kits

| | Erwinia | Phytophthora | Plant symptom |
|---|---|---|---|
| High | 10⁶ cfu/g | 10⁵ cfu/g | Development stage |
| potency | or higher | or higher | of onset |
| Medium | 10⁵ cfu/g | 10⁴ cfu/g | Middle stage |
| potency | | | of onset |
| Low | 10⁴ cfu/g | 10³ cfu/g | Early stage |
| potency | or lower | or lower | of onset |
| Negative | 10³ cfu/g | 10² cfu/g | No symptoms |
| | or lower | or lower | |

Referring to Table 5, each cut-off level was determined as low potency: 10⁴ cfu/g, medium potency: 10⁵ cfu/g, and high potency: 10⁶ cfu/g in the case of Erwinia, and low potency: 10³ cfu/g, medium potency: 10⁴ cfu/g, and high potency: 10⁵ cfu/g in the case of Phytophthora. In the case of the high potency and medium potency, the pathogens present in the soil were determined to be a high density (high risk period) just before the onset of disease that may immediately cause the disease in plants, and in the case of the low potency, the pathogens were proliferated, but were determined as a low density (low risk period) that did not immediately cause the disease. At this time, Erwinia was determined as negative in the case of 10³ cfu/g, and Phytophthora was determined as negative in the case of 10² cfu/g.

### Example 4

### Efficacy Test of Erwinia Diagnostic Strip According to the Present Invention

A. Domestic farmhouse soil samples provided from ABC Circle, Co., Ltd. were diagnosed by using the Erwinia diagnosis kit prepared in Example 2. The samples were confirmed for infection using 26 positive samples and 34 negative samples in plantation soil having a bacterial soft rot disease. The infection was determined by measuring the density of pathogens through microbial isolation and identification, which was shown in Table 6 below.

TABLE 6
Performance comparison of Erwinia and Phytophthora diagnosis kits

| | | Isolation | | Isolation | | |
|---|---|---|---|---|---|---|
| | | and | | and | | |
| | | identifica- | Erwinia | identifica- | Agdia | Phytophthora |
| | | tion of | Kit of the | tion of | Pytophthora | Kit of the |
| | | Erwinia | present | Phytophthora | Immuno- | present |
| No. | Samples | (cfu/g) | invention | (cfu/g) | Strip | invention |
| 1 | Tomato | ≤10³ | Negative | 2.5 × 10³ | Positive | Low |
| | | | | | | potency |
| 2 | Tomato | ≤10³ | Negative | 9.0 × 10⁴ | Positive | Medium |
| | | | | | | potency |
| 3 | Tomato | ≤10³ | Negative | 1.0 × 10³ | Positive | Low |
| | | | | | | potency |
| 4 | Tomato | 4.0 × 10⁵ | Medium | ≤10² | Negative | Negative |
| | | | potency | | | |
| 5 | Tomato | 1.0 × 10⁴ | Negative | ≤10² | Negative | Negative |
| 6 | Tomato | 4.5 × 10⁴ | Low potency | ≤10² | Negative | Negative |
| 7 | Tomato | 7.0 × 10⁴ | Low potency | ≤10² | Negative | Negative |
| 8 | Tomato | 2.0 ×10⁵ | Medium | ≤10² | Negative | Negative |
| | | | potency | | | |
| 9 | Chinese | 8.0 ×10⁶ | High | ≤10² | Negative | Negative |
| | cabbage | | potency | | | |
| 10 | Chinese | 2.5 × 10⁵ | Medium | ≤10² | Negative | Negative |
| | cabbage | | potency | | | |
| 11 | Chinese | 1.5 × 10⁴ | Low potency | ≤10² | Negative | Negative |
| | cabbage | | | | | |
| 12 | Chinese | 5.5 × 10⁴ | Low potency | ≤10² | Negative | Negative |
| | cabbage | | | | | |
| 13 | Chinese | 4.0 × 10⁴ | Low potency | ≤10² | Negative | Negative |
| | cabbage | | | | | |
| 14 | Potato | ≤10³ | Negative | 2.5 × 10⁴ | Positive | Medium |
| | | | | | | potency |
| 15 | Potato | ≤10³ | Negative | 1.0 × 10³ | Positive | Low |
| | | | | | | potency |
| 16 | Potato | ≤10³ | Negative | 5.5 × 10³ | Positive | Low |
| | | | | | | potency |
| 17 | Potato | ≤10³ | Negative | 4.0 × 10³ | Positive | Low |
| | | | | | | potency |
| 18 | Potato | ≤10³ | Negative | 2.0 × 10⁵ | Positive | High |
| | | | | | | potency |
| 19 | Potato | ≤10³ | Negative | 6.0 × 10³ | Positive | Negative |
| 20 | Potato | ≤10³ | Negative | 4.5 × 10³ | Positive | Low |
| | | | | | | potency |
| 21 | Potato | ≤10³ | Negative | 2.5 × 10⁴ | Positive | Medium |
| | | | | | | potency |
| 22 | Potato | ≤10³ | Negative | 8.0 × 10³ | Positive | Low |
| | | | | | | potency |
| 23 | Tomato | 3.5 × 10⁶ | High | ≤10² | Negative | Negative |
| | | | potency | | | |
| 24 | Tomato | 8.0 ×10⁵ | Medium | ≤10² | Negative | Negative |
| | | | potency | | | |
| 25 | Tomato | 2.0 ×10⁶ | High | ≤10² | Negative | Negative |
| | | | potency | | | |
| 26 | Tomato | 3.0 × 10⁶ | High | ≤10² | Negative | Negative |
| | | | potency | | | |
| 27 | Tomato | 6.5 × 10⁵ | Medium | ≤10² | Negative | Negative |
| | | | potency | | | |
| 28 | Chinese | 1.5 × 10⁴ | Low potency | ≤10² | Negative | Negative |
| | cabbage | | | | | |
| 29 | Chinese | 5.5 × 10⁵ | Medium | ≤10² | Negative | Negative |
| | cabbage | | potency | | | |
| 30 | Chinese | 2.5 ×10⁴ | Low potency | ≤10² | Negative | Negative |
| | cabbage | | | | | |
| 31 | Chinese | ≤10³ | Negative | 5.5 × 10³ | Positive | Low |
| | cabbage | | | | | potency |
| 32 | Chinese | ≤10³ | Negative | 4.5 × 10⁴ | Positive | Medium |
| | cabbage | | | | | potency |
| 33 | Chinese | ≤10³ | Negative | 8.0 × 10⁵ | Positive | High |
| | cabbage | | | | | potency |
| 34 | Tomato | 3.5 × 10⁴ | Low potency | ≤10² | Negative | Negative |
| 35 | Tomato | 2.0 × 10⁴ | Low potency | ≤10² | Negative | Negative |
| 36 | Tomato | 2.5 × 10⁴ | Medium | ≤10² | Negative | Negative |
| | | | potency | | | |
| 37 | Tomato | 5.5 × 10⁴ | Medium | ≤10² | Negative | Negative |
| | | | potency | | | |
| 38 | Tomato | 3.5 × 10⁶ | High | ≤10² | Negative | Negative |
| | | | potency | | | |
| 39 | Tomato | 1.0 × 10⁶ | High | ≤10² | Negative | Negative |
| | | | potency | | | |
| 40 | Potato | ≤10³ | Negative | 1.5 × 10³ | Positive | Low |
| | | | | | | potency |
| 41 | Potato | ≤10³ | Negative | 4.0 × 10³ | Positive | Low |
| | | | | | | potency |
| 42 | Potato | ≤10³ | Negative | 5.0 × 10³ | Positive | Low |
| | | | | | | potency |
| 43 | Potato | ≤10³ | Negative | 2.5 × 10⁴ | Positive | Medium |
| | | | | | | potency |
| 44 | Potato | ≤10³ | Negative | 2.0 × 10⁴ | Positive | Medium |
| | | | | | | potency |
| 45 | Potato | ≤10³ | Negative | 5.0 × 10⁵ | Positive | High |
| | | | | | | potency |
| 46 | Potato | ≤10³ | Negative | 4.5 × 10⁵ | Positive | High |
| | | | | | | potency |
| 47 | Potato | 8.5 × 10⁴ | Low potency | ≤10² | Negative | Negative |
| 48 | Potato | 4.5 × 10⁴ | Low potency | ≤10² | Negative | Negative |
| 49 | Chinese | ≤10³ | Negative | ≤10² | Negative | Negative |
| | cabbage | | | | | |
| 50 | Chinese | ≤10³ | Negative | ≤10² | Negative | Negative |
| | cabbage | | | | | |
| 51 | Chinese | ≤10³ | Negative | ≤10² | Negative | Negative |
| | cabbage | | | | | |
| 52 | Chinese | ≤10³ | Negative | ≤10² | Negative | Negative |
| | cabbage | | | | | |
| 53 | Chinese | ≤10³ | Negative | ≤10² | Negative | Negative |
| | cabbage | | | | | |
| 54 | Chinese | ≤10³ | Negative | ≤10² | Negative | Negative |
| | cabbage | | | | | |
| 55 | Chinese | ≤10³ | Low potency | ≤10² | Negative | Negative |
| | cabbage | | | | | |
| 56 | Chinese | ≤10³ | Negative | ≤10² | Negative | Negative |
| | cabbage | | | | | |
| 57 | Chinese | ≤10³ | Negative | ≤10² | Negative | Negative |
| | cabbage | | | | | |
| 58 | Chinese | ≤10³ | Negative | ≤10² | Negative | Negative |
| | cabbage | | | | | |
| 59 | Chinese | ≤10³ | Negative | ≤10² | Negative | Negative |
| | cabbage | | | | | |
| 60 | Chinese | ≤10³ | Negative | ≤10² | Negative | Negative |
| | cabbage | | | | | |

* Measurement was not made accurately below Erwinia microbial density of 10³ cfu/m or lower
* Measurement was not made accurately below Phytophthora microbial density of 10² cfu/m or lower

The examination was performed by the diagnosis kit according to the present invention and the microbial isolation and identification method, and the relative sensitivity and relative specificity of the diagnosis kit were shown in Table 7 by measuring the number of detected samples.

TABLE 7
Relative sensitivity and relative specificity of Erwinia diagnosis
kit and microbial isolation and identification method

| | | Microbial isolation and identification | | | | |
|---|---|---|---|---|---|---|
| | | (Erwinia) | | | | |
| | | | | | Negative | |
| | | ≥10⁶ | 10⁵ | ≥10⁴ | ≥10³ | |
| | | cfu/g | cfu/g | cfu/g | cfu/g | Total |
| Kit of the | High potency | 6 | — | — | — | 6 |
| present | ≥10⁶ cfu/g | | | | | |
| invention | Medium | — | 8 | — | — | 8 |
| | potency = | | | | | |
| | 10⁵ cfu/g | | | | | |
| | Low potency | — | — | 11 | 1 | 12 |
| | ≤10⁴ cfu/g | | | | | |
| | Megative | — | — | 1 | 33 | 34 |

| | | | | | |
|---|---|---|---|---|---|
| Total | 6 | 8 | 12 | 34 | 60 |

| | |
|---|---|
| High potency | 100% |
| relative sensitivity | |
| Medium potency | 100% |
| relative sensitivity | |
| Low potency | 91% |
| relative sensitivity | |
| Relative specificity | 97% |

In Table 7, the relative sensitivity by potency refers to a ratio of samples whose pathogen density was confirmed through the microbial isolation and identification method to diagnose the same potency in the Erwinia diagnosis kit according to the present invention, and the relative specificity refers to a ratio of negatively confirmed samples to test negative in the diagnosis kit. As a result, the relative sensitivity by potency showed high potency 100%, medium potency 100%, low potency 91%, and the relative specificity showed 97%. Therefore, it could be seen that the performance of the diagnosis kit according to the present invention showed excellent results.

### Example 5

### Efficacy Test of Phytophthora Diagnostic Strip According to the Present Invention

A. The examination was performed by the diagnosis kit according to the present invention and the microbial isolation and identification method, each was examined with an Agdia Phytophthora ImmunoStrip test, and the relative sensitivity and relative specificity of the diagnosis kit were shown in Tables 8 and 9 by measuring the number of detected samples.

The relative sensitivity refers to a ratio of samples confirmed positive in the Agdia Phytophthora ImmunoStrip Test to test positive in the diagnosis kit according to the present invention, and the relative specificity refers to a ratio of negatively confirmed samples to test negative in the diagnosis kit.

As a result, the same results were confirmed in 59 of 60 samples compared to the Agdia Phytophthora ImmunoStrip Test to have the relative sensitivity of 95% and the specificity of 100%.

The relative sensitivity by potency refers to a ratio of samples whose pathogen density was confirmed through the microbial isolation and identification method to diagnose the same potency in the Phytophthora diagnosis kit according to the present invention, and the relative specificity refers to a ratio of negatively confirmed samples to test negative in the diagnosis kit.

As a result, the relative sensitivity by potency showed high potency 100%, medium potency 100%, low potency 91%, and the relative specificity showed 100%. Therefore, it could be seen that the performance of the diagnosis kit according to the present invention showed excellent results.

TABLE 8
Relative sensitivity and Relative specificity between
Phytophthora diagnosis kit and other products

Agdia Phytophthora
ImmunoStrip Test

| | | Positive | Negative | Total |
|---|---|---|---|---|
| 60 samples | Positive | 21 | — | 21 |
| | Negative | 1 | 38 | 39 |

| | | | |
|---|---|---|---|
| Total | 22 | 38 | 60 |

| | |
|---|---|
| Relative sensitivity | 95% |
| Relative specificity | 100% |

TABLE 9
Relative Sensitivity and Relative Specificity of Pytophthora Diagnosis
kit and Microbial Isolation and Identification

| | | Microbial Isolation and Identification | | | | |
|---|---|---|---|---|---|---|
| | | (Pytophthora) | | | | |
| | | | | | Negative | |
| | | ≥10⁵ | =10⁴ | ≥10³ | ≥10² | |
| | | cfu/g | cfu/g | cfu/g | cfu/g | Total |
| Kit of the | High potency | 4 | — | — | — | 4 |
| present | ≥10⁵ cfu/g | | | | | |
| invention | Medium | — | 6 | — | — | 6 |
| | potency = | | | | | |
| | 10⁴ cfu/g | | | | | |
| | Low potency | — | — | 11 | — | 11 |
| | ≤10³ cfu/g | | | | | |
| | Negative | — | — | 1 | 38 | 39 |

| | | | | | |
|---|---|---|---|---|---|
| Total | 4 | 6 | 12 | 38 | 60 |

| | |
|---|---|
| High potency | 100% |
| relative sensitivity | |
| Medium potency | 100% |
| relative sensitivity | |
| Low potency | 91% |
| relative sensitivity | |
| Relative specificity | 100% |

As described above, the technical ideas for the simultaneous diagnosis method for Erwinia carotovora and Phytophthora infestans using the semi-quantitative lateral flow immunoassay according to the present invention and the antibodies used herein have been specifically described in the preferred embodiment, but it should be noted that the embodiment is for explanation, not for limitation. In addition, it is obvious to those skilled in the art that various variations and modifications are possible within the scope of the technical spirit of the present invention, and therefore, it is natural that these variations and modifications fall within the scope of the appended claims.

### EXPLANATION OF REFERENCE NUMERALS AND SYMBOLS

1: Immunoassay device
2: Assay strip
3: Lower case
3A, 3B: First strip support, second strip support
4: Upper case
4A, 4B: First strip counterpart, second strip counterpart
21: Sample pad
221: Test line
222: Control line
22: Membrane
23: Absorbent pad
24: Short side
25: Long side
31: Short side fixing part
32: Long side fixing part
33: Lower blocking part
41: Sample inlet
42: Text indicator
42: Result display window

1. A simultaneous diagnosis method for Erwinia carotovora and Phytophthora infestans in a soil using semi-quantitative lateral flow immunoassay comprising:
providing a rat monoclonal antibody for each of Erwinia carotovora (Erwinia) causing a bacterial soft rot disease and Phytophthora infestans (Phytophthora) causing a fungal blight disease;
binding Erwinia and Phytophthora to the respective rat antibodies; and
quantifying amounts of the bound Erwinia and Phytophthora,
wherein in the providing of the rat monoclonal antibody of each of Erwinia and Phytophthora,
rats are immunized using each of the Erwinia carotovora (Erwinia) and Phytophthora infestans (Phytophthora) as antigens and inactivating the Erwinia carotovora (Erwinia) and Phytophthora infestans (Phytophthora) at a high temperature to be used as immunogens, a hybridoma is prepared by cell-fusing rat splenocytes and myeloma cells (Sp2/0 Ag-18) at a ratio of splenocytes:myeloma cells of 5:1, in the hybridoma, monoclonal antibodies, which are specific for Erwinia and Phytophthora, respectively, and do not show cross-reactivity to bacteria Lactobacillus plantarum, Bacillus subtillis, Rhodobacter capsulatus, Saccharomyces cerevisiae, Bacillus velezensis, and Bacillus thuringiensis, and fungi Fusarium oxysporum, Mycosphaerella nawae, Rhizoctonia solani and Colletotrichum gloeosporioides, respectively, are obtained, and rat anti-Erwinia antibody and anti-Phytophthora antibody are selected from the obtained antibodies,
wherein the rat anti-Erwinia 5E10 antibody is added to a gold solution while stirring to be a final concentration of 14 ug/ml, the rat anti-Phytophthora 1C6 antibody is added to the gold solution while stirring to be a final concentration of 20 ug/ml, and each gold particle suspension is added with a 10% BSA solution and stirred, and then the bound gold solution is centrifuged, to remove unbound antibodies, a supernatant is discarded, and 5 mM borate (sodium tetraborate) added with 1% BSA 3 times larger than the volume of a pellet is added to the bound gold solution (pellet), and then the pellet is resuspended, and the suspension is centrifuged again, an antibody gold conjugate is prepared by adding the final pellet to 5 mM borate (sodium tetraborate) added with 1% BSA and adjusting an absorbance 10±1 O.D at 530 nm with a spectrophotometer, and finally, a rat anti-Erwinia 5E10 antibody gold conjugate and a rat anti-Phytophthora 1C6 antibody gold conjugate are obtained, respectively.

2. The simultaneous diagnosis method for Erwinia carotovora and Phytophthora infestans in a soil using semi-quantitative lateral flow immunoassay of claim 1, wherein
in the quantifying of the amounts of Erwinia carotovora and Phytophthora infestans, causative organisms of a bacterial soft rot disease and a fungal blight disease of plants are determined simultaneously by selecting cut-off levels of a detection kit and semi-quantitatively analyzing an infection source using the detection kit applied with the rat anti-Erwinia antibody and the rat anti-Phytophthora antibody,
wherein each cut-off level is determined as low potency: 10⁴ cfu/g, medium potency: 10⁵ cfu/g, and high potency: 10⁶ cfu/g in the case of Erwinia, and low potency: 10³ cfu/g, medium potency: 10⁴ cfu/g, and high potency: 10⁵ cfu/g in the case of Phytophthora, wherein in the case of the high potency and medium potency, pathogens present in the soil are determined to be a high density (high risk period) just before onset of disease that may immediately cause the disease in plants, and in the case of the low potency, the pathogens are determined as a low density (low risk period) in which the pathogens are proliferated but do not immediately cause the disease, wherein Erwinia is determined as negative in the case of 10³ cfu/g, and Phytophthora is determined as negative in the case of 10² cfu/g.

3. The simultaneous diagnosis method for Erwinia carotovora and Phytophthora infestans in a soil using semi-quantitative lateral flow immunoassay of claim 1, wherein
the rat anti-Erwinia antibody and the rat anti-Phytophthora antibody produced by a hybridoma cell line are rat monoclonal antibodies showing specific immunoreactivity for Erwinia carotovora and Phytophthora infestans, respectively, and the rat monoclonal antibodies do not show cross-reactivity with each other.

4. The simultaneous diagnosis method for Erwinia Carotovora and Phytophthora Infestans in a soil using semi-quantitative lateral flow immunoassay of claim 1, wherein
the obtained rat anti-Erwinia antibody and rat anti-Phytophthora antibody are mass-produced using a serum-free culture medium production method to mass-produce the antibodies in the hybridomas, and
serum free media without containing animal-derived serum are used for the serum-free culture medium in which the hybridomas are cultured, sodium hypoxanthine and thymidine, which are used in a de novo biosynthesis pathway of nucleoside, as additives and penicillin-streptomycin as an antibiotic are used, and amino acids, vitamins, glucose, glutamine, trace elements, growth factors, insulin, transferrin, selenium, and pyruvate are used as nutritional supplements.

5. A simultaneous diagnosis kit for Erwinia carotovora and Phytophthora infestans in a soil using semi-quantitative lateral flow immunoassay comprising:
a primary antibody for capturing Erwinia carotovora bacterial and Phytophthora infestans fungal antigens, a detection label, a secondary antibody bound with the detection label, and a reagent for measuring an activity of the detection label,
wherein the primary antibody and the secondary antibody for capturing the antigens are used with a rat anti-Erwinia 5E10 antibody gold conjugate and a rat anti-Phytophthora 1C6 antibody gold conjugate obtained in the step of providing the rat monoclonal antibody to each of Erwinia and Phytophthora in the simultaneous diagnosis method of claims 1, and
a sample to be used is a soil diluent.
